(19) United States
(12) Patent Application Publication
(10) Pub. No.: US 2009/0175963 A1
(43) Pub. Date: Jul. 9, 2009

(54) ANTIVIRAL COMPOSITION COMPRISING LYCORIS SQUAMIGERA EXTRACTS

(76) Inventors: Hyuk Joon Kwon, Seoul (KR); Sun Hee Cho, Gyeonggi-do (KR); Sun Joong Kim, Gyeonggi-do (KR); Young Jin Ahn, Gyeonggi-do (KR); Jeong Chan Ra, Gyeonggi-do (KR)

Correspondence Address: INTELLECTUAL PROPERTY / TECHNOLOGY LAW, PO BOX 14329, RESEARCH TRIANGLE PARK, NC, 27709, US

(21) Appl. No.: 11/570,041

(22) PCT Filed: Aug. 29, 2006
(86) PCT No.: PCT/KR2006/003403

§ 371 Date: Dec. 21, 2006

(30) Foreign Application Priority Data
Jun. 29, 2006 (KR) 10-2006-0059182

(51) Int. Cl. A61K 36/185; A61P 31/16

(52) U.S. Cl. 424/725

(57) ABSTRACT

Antiviral compositions including Lycoris squamigera extracts are described as useful for preventing or treating diseases caused by influenza virus infection of humans and other mammalian and avian subjects (e.g., pigs, horses, birds, and the like). Lycoris squamigera extracts in such use exhibit low toxicity in normal cell environments, and excellent antiviral effects. Compositions and anti-viral agents for influenza virus that include Lycoris squamigera extracts are effectively used in foods and pharmaceutical products for preventing and treating influenza virus diseases.

### TECHNICAL FIELD

The present invention relates to an antiviral composition comprising Lycoris squamigera extracts, more specifically, relates to a composition for preventing or treating diseases caused by influenza virus which infects human, pig, horse, bird, and the like.

### BACKGROUND ART

Virus cause various diseases, particularly, a typical one among pathogenic viruses that become a problem in the field of stockbreeding is Avian influenza virus. Avian influenza virus belongs to the orthomixoviridae family, and cause damage to poultry such as chicken, turkey. Avian influenza virus is classified into 3 types of high-pathogenic, low-pathogenic and non-pathogenic Avian influenza viruses according to the degree of pathogenicity, among which the high-pathogenic is classified into “grad A” in the World Organization for Animal Health (OIE) and “the first level domestic animal infectious disease” in Republic of Korea. Influenza virus is classified into type A.

The influenza virus is classified to A, B or C type according to the antigenicity of nucleocapsid protein and matrix protein. Moreover, according to the difference of antigen structure of haemagglutinin (HA) and neuraminidase (NA), the HA is classified to 16 subtypes and NA is classified to 9 subtypes, wherein HA helps a binding of host cell receptor, and a fusing between host cell membrane and viral envelope to cause a virus infection, wherein NA plays important role in when the virus buds from cell after proliferation. Theoretically, 144 kinds of virus subtypes could be existed by the combination of the proteins. The infection is generally occurred at contacting a secretion of birds, furthermore, spreaded by dejecta sticked at the surface of droplet, human feet, feed-stuff, car, apparatus and egg etc.

Although a symptom is various according to infected virus's pathogenicity, generally, the respiratory symptoms, diarrhea and a sharp decline of egg production ratio etc. are appeared. Moreover, in some cases, the cyanosis is appeared at crest of head, the edema is appeared in the face, or sometimes the phenomenon of gathering feathers is appeared. A mortality rate is also various within 0˜100% according to pathogenicity, but since the symptoms are similar to ones of Newcastle Disease, infectious larynogotracheitis, mycoplasma infection and the like, the accurate diagnosis is necessary.

It has been that high-pathogenic avian influenza is taken ill about 23 times in 1959 to 2003 all over the world, most of them were stamped out locally. H1N1 subtype high-pathogenic avian influenza generated in Korea in December 2003 is outbroken in more than 30 countries including Europe, Africa and most of Southeast Asia such like Japan, China, Thailand, Vietnam and Indonesia, that is, it showed a global aspect. Though it is known as avian influenza cannot be infected directly to human, the importance of public health about Avian Influenza virus is larger every day by the case of H5N1 infection to human body in 1997 at Honkong, H9N2 Avian Influenza separation from human body in 1999 and H7 infection to human body in 2004. According to report of the World Health Organization (WHO), (http://www.who.int/csr/disease/avian_influenza/country/cases_table_{—}2006_{—}06_{—}20/en/index.html), the 228 persons were infected with H1N1 subtype to the death in 2003 to Jun. 20, 2006 around 10 countries. In Korea, since low-pathogenic Avian Influenza by H9N2 subtype had been generated in 1996, it was re-generated in 1999 and it has been outbroken around the country from now.

If avian influenza is generated, most of countries all over the world treat them slaughtered, these countries cannot export the poultry products to bring swinging damages into poultry industry. Furthermore, when there is a risk of human body infection, the damages are spread to industry as a whole comprising a tourist industry and a transport industry, finally astronomical loss is incurred.

A natural substance means the thing in the raw, not added artificial factors, and the natural substance classified as GRAS (Generally Recognized As Safe) could be used without restriction of quantity or subject. At home industry, the natural substance classified as a nature additive, it has been used as food additive, and in foreign country, it has been used as health foods and medical supplies without extra limit for user's purpose, because of its excellent functionality.

The Lycoris squamigera is perennial grass of Amaryllidaceae, the original home is china and it is an ornamental plant. The bulb is wide egg-shaped, the diameter is about 4˜5 cm, and the outside color is deep brown having black.

The stem of a flower stand straight and its height is 50˜70 cm and slightly thick. In the spring, the leaf springs in a body from the end of bulb. The leaf grows with stripe shape by length of 20˜30 cm and width of 16˜25 cm and is dried up in the June˜July. The flower of Lycoris squamigera are in bloom in August and they are opened in umbel with 4˜8 flowers on the end of the stem of the flower. The involucre is divided into many pieces and each divided part is scarious and lanceolate with length of 2˜4 cm. The length of small spray of flowers is 1˜2 cm and that of flower is 9˜10 cm and its color is light purple incling to red.

The perianth of the flower has round shape in the bottom and is divided into 6 pieces in the top. Each divided part which is inverted lanceolate with length of 5˜7 cm is slightly leaned backward. In the flower, six stamina exist which are shorter than a perianth of the flower and the color of their anthers is light red. Also, in the flower, one pistil exist and an inferior ovary with trilocular has sterility. In the field of traditional oriental medicine, bulb of Lycoris squamigera is used as medicine and is known for alleviation of pain for infantile paralysis.

Many researchers through out the world give enormous endeavor to develop anti-viral agents at present. Lamibudine used in treatment of HIV (Human Immunodeficiency Virus)-1 and hepatitis B, gancyclovir used in treatment of herpes virus infection symptoms, ribavirin used in treatment of various virus infection symptoms, mostly respiratory syncytial virus infection symptoms and zanamivir Relenza™ and oseltamivir, TAMIFLU™ which are synthesized artificially as neuraminidase inhibitors of influenza virus are get an approval and are put on the market. However, use of amantadine and its analogue, rimantadine, which are approved for treatment of influenza virus A are reduced for appearance of resistant virus and its side effect. Recently, virus resistant to oseltamivir among H5N1 avian influenza virus appears, therefore, developments of various anti-virus agents are required.

Therefore, the present inventors had been studied the natural substance having a low toxicity in normal cell, while having an excellent effect to inhibition of proliferation of influenza virus. As a result, they discovered that a composition comprising Lycoris squamigera extracts have anti-influenza virus effects, and perfected the present invention.

### SUMMARY OF THE INVENTION

The present invention, in one aspect, relates to a food composition for preventing or treating viral diseases, comprising Lycoris squamigera extracts.

The present invention, in another aspect, relates to a pharmaceutical composition for preventing or treating viral diseases, comprising Lycoris squamigera extracts.

Other features and examples of the invention will be clarified from the minute descriptions and appended claims as follows.

### DETAILED DESCRIPTION OF THE INVENTION, AND PREFERRED EMBODIMENTS

In the present invention, after a composition containing Lycoris squamigera extracts was added to SPF embryonated egg infected with Avian influenza virus and cultured, the plate hemagglutination test was performed, and as a result, it was confirmed that the composition containing Lycoris squamigera extracts has excellent anti-viral effect.

Accordingly, the present invention provides a food composition for preventing or treating influenza viral diseases belonging to the orthomixoviridae family, comprising the Lycoris squamigera extracts and a sitologically acceptable supplemental additive

The present invention also provides a pharmaceutical composition for preventing or treating the influenza virus diseases belonging to the orthomixoviridae family, comprising Lycoris squamigera extracts as an active ingredient.

In the present invention, said influenza virus is preferably selected from the group consisting of: human influenza virus, Swine influenza virus, Equine influenza virus, and Avian influenza virus. More preferably, said Avian influenza virus is KBNP-0028 (KCTC 10866BP).

### EXAMPLES

Hereinafter, the present invention will be described in more detail by examples. However, it is obvious to a person skilled in the art that these examples are for illustrative purpose only and are not construed to limit the scope of the present invention.

### Example 1

### Preparation of Lycoris squamigera Extracts

The leaves, underground parts, and whole shoot of Lycoris squamigera were picked, dried at room temperature for 24 hrs, chopped up and crushed. The obtained powder was added with 99.9% methanol, stirred for 24 hrs at room temperature to extract, vacuum-filtered to collect supernatant liquid and eluted useful components from the obtained powder. The useful components are dried for 24 hrs at room temperature, and dissolved in 99.9% dimethyl sulfoxide (DMSO) solution to 20 mg/ml.

Although the Lycoris squamigera extracts of the present invention could be obtained by the above described method, herein it is distributed from The Korea Plant Extract Bank to use.

### Example 2

### Examination of Anti-Viral Effect of Lycoris squamigera Extracts

### 2-1: Preparation of KBNP-0028

As avian influenza virus used in the present invention, KBNP-0028 (KR 2006-0026591) cloned after subculturing A/chicken/Korea/SNU0028/2000(H9N2) virus (it is separated in Korea in 2000) in chick embryo was used. That is, SNU0028 [A/chicken/Korea/SNU0028/2000(H9N2); separation and declaration to National Veterinary Research and Quarantine Service, May 9, 2005] is low-pathogenic Avian Influenza virus of H9N2 subtype, separated from chicken showing mortality and egg drop syndrome. The virus was separated in a chicken farm located in North jeola Province in Jan. 28, 2000.

The separating method is as follows: after kidney and tracheal sample from infected chicken are dissolved, suspended in phosphate buffer, and filterated with 0.45 μm diameter filter paper, each sample is inoculated into three allantoic cavities of SPF (Specific Pathogen Free) embryonated egg (Sunrise Co., NY), and cultured at 37° C. to obtain allantoic fluid. The 20 ml of allantoic fluid and 20 μl of 0.1% chicken red blood cells extracted from a chicken obtained hatching the SPF embryonated egg are dropped on glass plate, and mixed to carry out the plate hemagglutination test.

As a result, all of the allantoic fluids obtained by inoculating kidney sample and tracheal sample formed the hemagglutination. The virus was identified with RT-PCR and the analysis of base sequence using H9N2 specific primer (Kim Min Chul, Master's Thesis, 2002, Seoul National University), and stored at −70° C. Among them, the virus separated from tracheal sample was used in the present invention.

In order to select a vaccinia strain having high-productivity of embryonated egg, the separated SNU0028 was diluted with phosphate buffer to the concentration of 0.05 to 0.5 HAU/ml. 200 μl of diluted solution was inoculated to 10-11-day-old SPF hatchery egg (Sunrise Co., NY) via the allantoic cavity, and cultured for three days at 37° C. Everyday, the embryonated eggs, which died three days ago, was discarded through egg examination in the morning and afternoon.

The embryonated egg, which survived for three days was, stored for 12˜24 hrs at 4° C., from which allantoic fluid was collected to measure each of volume and hemagglutination titer thereof. Among them, allantoic fluid having the most quantity and the highest hemagglutination titer was inoculated to embryonated egg with the same method as described above, and subcultured 19 times to separate eggs whose productivity was increased, due to high hemagglutination titer high yield of allantoic fluid and thus they are named KBNP-0028. It is deposited at GenBank located Eoeundong, Youseonggu, Daejeon city, Korea on Oct. 26, 2005 (KCTC 10866BP).

### 2-2: Culturing Hatchery Egg Shell Fragments

The egg shell of 10-11 day-old SPF hatchery egg (Sunrise Co., NY) was washed with 70% ethanol, and all of the chick embryo and body fluid were removed. The resulting egg shell is cut into about 8 mm long and 8 mm wide while maintaing villi, allantois adhere to the interior of egg shell, and put them in 24 well culture medium piece by piece. The culture medium was prepared by (i) mixing 199 medium (GIBCO-BRL, NY, USA) with F10 medium (GIBCO-BRL, NY, USA) at a ratio of 1:1, (ii) adding 0.075% of sodium bicarbonate and 100 μg/Ml of gentamicin.

To the 10-11-day-old SPF embryonated egg (Sunrise Co., NY) was infected with virus by adding 100 μl of crude allantoic fluid KBNP-0028 prepared in Example 2-1, which is 4˜10-fold diluted to the surface of villi allantois of hatchery egg shell fragments, and culturing for 30 min at 37° C. 1000 μl of the culture medium was added to the infected egg, and then Lycoris squamigera extracts was added to 6 well plates respectively to the concentration of 400, 300, 200, 100, 50 and 12.5 μg/Ml. The virus-infected fluid containing Lycoris squamigera extracts was cultured for 7 days at 37° C.

### 2-3: Test of Antiviral Effects

Culture broth of said virus-infected fluid containing Lycoris squamigera extracts, prepared in Example 2-2 was collected to carry out plate hemagglutination test. 25 μl of the culture broth and 25 μl of chicken red blood cells (0.1%) were dropped on glass plate in the same amount and mixed evenly. The virus proliferation was examined according to by whether hemagglutination was formed within 2 min after moving the glass plate right and left, and up and down. As a result, in case of the leaves, virus proliferation was completely inhibited until the concentration reached 300 μg/Ml without toxicity in cell, and showed partial antiviral effect at concentration of 200 μg/Ml. In the case of underground parts, it showed partial antiviral effect even at concentration of 12.5 μg/Ml. Additionally, the whole shoot showed complete virus inhibition effect until concentration reached 50 μg/Ml, and partial virus inhibition effect until concentration reached 12.5 μg/Ml (Table 1).

### 2-4: MTT Assay

Lycoris squamigera (scientific name: Lycoris squamigera, generic name: Amaryllidaceae, family name: Amaryllidaceae) extracts prepared in example 2-2 was put into 6 well plates of 400, 300, 200, 100, 50 and 12.5 μg/Ml added with 40 μl of MTT (3-(4,5-dimethylthiazol-2-yl)-2,5-diphenyl tetrazolium bromide) solution (MTT 0.5% aqueous solution), respectively and cultured for 1˜3 hrs at 37° C. 120 μl of DMSO was added and stirred for 30 min, then the result was read at 562 nm wavelength with ELISA (Table 1). As a result, it was confirmed that the measured value has no significant difference compared to the MTT value of the control group added only with virus (0.381±0.057), and there was no cytotoxicity by extracts

TABLE 1
MTT Assay result according to the part of Lycoris squamigera

| | Extract concentration (μg/ml) | | | | | | | | |
|---|---|---|---|---|---|---|---|---|---|
| | HA positive (MTT OD mean ± standard deviation) | | | | | | | Control | |
| Site | 400 | 300 | 200 | 100 | 50 | 25 | 12.5 | Virus | Nonvirus |
| Leaf | 0/6 | 0/6 | 2/6 | 6/6 | NT | NT | NT | 6/6 | 0/6 |
| | (0.316 ± 0.060) | (0.303 ± 0.059) | (0.273 ± 0.053) | (0.379 ± 0.070) | | | | (0.381 ± 0.057) | (0.403 ± 0.118) |
| Underground | 0/3 | 0/3 | 0/3 | 0/3 | 0/3 | 0/3 | 0/3 | | |
| parts | (0.284 ± 0.051) | (0.244 ± 0.036) | (0.332 ± 0.079) | (0.324 ± 0.067) | | | | | |
| Whole | 0/3 | 0/3 | 0/3 | 0/3 | 0/3 | 1/3 | 2/3 | | |
| shoot | (0.298 ± 0.024) | (0.298 ± 0.069) | (0.316 ± 0.017) | (0.363 ± 0.042) | | | | | |

NT: No tested

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described above in detail, the Lycoris squamigera extracts according to the present invention have a low toxicity in choriollantonic cell which is a normal cell, while having excellent antiviral effect. Therefore, the composition comprising Lycoris squamigera extracts can be used effectively in food and pharmaceutical composition since it is effective and safe in preventing and treating influenza virus diseases.

1. A composition of foods for preventing influenza virus diseases, comprising Lycoris squamigera extracts and a sitologically acceptable supplemental additive.

2. The composition according to claim 1, wherein said influenza virus is any one selected from the group consisting of: human influenza virus, Swine influenza virus, Equine influenza virus, and Avian influenza virus.

3. The composition according to claim 2, wherein said Avian influenza virus is KBNP-0028 (KCTC 10866BP)

4. A pharmaceutical composition for preventing or treating influenza virus diseases, comprising Lycoris squamigera extracts as an active ingredient.

5. The pharmaceutical composition according to claim 4, wherein said influenza virus is any one selected from the group consisting of: human influenza virus, Swine influenza virus, Equine influenza virus, and Avian influenza virus.

6. The pharmaceutical composition according to claim 5, wherein said Avian influenza virus is KBNP-0028 (KCTC 10866BP).

7. An anti-viral agent for influenza virus, comprising an Lycoris squamigera extract as an active ingredient.

8. The anti-viral agent for influenza virus of claim 7, wherein the influenza virus is any one of: human influenza virus, Swine influenza virus, Equine influenza virus, and Avian influenza virus.
